(19) United States
(12) Patent Application Publication
(10) Pub. No.: US 2008/0177477 A1
(43) Pub. Date: Jul. 24, 2008

(54) Compositions and Methods Comprising Biological Samples for Quality Controls

(75) Inventors: John Rader, Dallas, TX (US); Rick Staub, Dallas, TX (US)

Correspondence Address: KALOW & SPRINGUT LLP, 488 MADISON AVENUE, 19TH FLOOR, NEW YORK, NY, 10022, US

(73) Assignee: ORCHID CELLMARK INC, Princeton, NJ (US)

(21) Appl. No.: 11/664,659

(22) PCT Filed: Oct. 5, 2005
(86) PCT No.: PCT/US05/35949

§ 371 Date: Feb. 13, 2008

Related U.S. Application Data
(60) Provisional application No. 60/616,398, filed on Oct. 5, 2004.

(51) Int. Cl. C12Q 1/68; G01N 33/68; C12Q 1/04; G06F 17/30

(52) U.S. Cl. 702/19; 435/6; 435/7.1; 435/34; 707/104.1; 707/E17.005

(57) ABSTRACT

A quality control system for testing biological samples is provided, comprising: a support having at least one surface capable of receiving one or more predetermined quantities of a biological sample from one or more known donors. A computerized system for testing biological samples is also provided.

This application claims the benefit of the filing date of U.S. Provisional Patent Application No. 60/616,398 filed Oct. 5, 2004, entitled “Compositions And Methods Comprising Biological Samples For Quality Controls”, this entire disclosure is hereby incorporated by reference into the present disclosure.

### BACKGROUND OF THE INVENTION

Numerous methods and systems have been developed for conducting biological assays that are essential in a variety of applications including, medical diagnostics, genotyping, paternity and genetic or forensic identification, testing of foods, environmental monitoring, and basic scientific research. Depending on the application, it is desirable that the biological assay has high sensitivity, precision, reliability, and accuracy.

Critical to the reliability and accuracy of the biological assays is the use of appropriate control or validation samples. Biological controls assist the researcher in providing accurate, precise data with a high level of confidence that the questioned biological specimen analyzed is free from contamination and that errors and variability of test results on the unknown sample are minimized or avoided by comparison to the results obtained from a known or unknown control sample.

Typically, internal quality control methods are utilized for biological assays. Internal quality control involves assaying the unknown sample against the control sample using the same analyzer, under the same conditions and monitoring whether reliable and predictable assay results are obtained.

When dealing with genetic or forensic identification, great strides have been made toward systems capable of identifying the source of a biological sample containing nucleic acids with a high degree of sensitivity, precision, reliability, and accuracy. A wide variety of nucleic acid analysis techniques are available for applications aimed at revealing genetic similarities between samples of nucleic acids. For example, highly polymorphic repetitive sequences that exist in genomes may be employed in genetic identification applications. These applications allow for identification or differentiation of individuals in a population with a high degree of confidence. One important application relies upon the analysis of polymorphic tandem repeat loci. One example of a genetic identification application is the FBI's Combined DNA Indexing System, or CODIS, which employs thirteen polymorphic short tandem repeat loci for genetic identification.

Tandem repeat loci are loci in a genome that contain repeat units of nucleotide sequences of varying length, such as dinucleotide repeats, trinucleotide repeats, tetranucleotide repeats, and so forth. The length of the repeating unit varies from as small as two nucleotides to extremely large numbers of nucleotides. The repeats may be simple tandem sequence repeats or complex combinations thereof. Variations in the length or character of these repeats at such loci are referred to as polymorphisms at these loci. These polymorphisms most frequently arise through the existence of varying numbers of these repeats at a locus between individuals in a population.

By some estimates, tandem repeats are encountered in the human genome at an average frequency of about 15 kilobases. The number of alleles, or varieties of sequence repeats at a locus, typically vary from about as few as three or four to as many as fifteen or up to fifty or more. Their relative high frequency of occurrence, coupled with their significant degree of polymorphism, render these features of the genome attractive candidates for genetic identification applications. By examining a sufficient number of polymorphic tandem repeat loci in a sample of nucleic acids and comparing the characteristics of the loci of that individual with the characteristics of the same loci in a reference sample from a second individual, a determination can be made as to whether the individual is genetically related to the second individual from whom the reference sample was obtained. Generally, polymorphic repeat loci employed in genetic or forensic identification applications are selected so as to be unlinked, or in Hardy-Weinberg equilibrium, with one another.

Various types of tandem repeat loci are employed in genetic or forensic identification applications. Short tandem repeats (STRs) arise from variations in the number of short stretches of nucleic acid sequences. In the human genome, STRs are believed to occur about once in every few hundred thousand bases. STRs span about 2-7 bases, and vary with respect to the number of repeat units they contain and exist as both simple and complex repeats. Another type of tandem repeat, minisatellite repeats, are usually about 10 to 50 or so bases repeated about 20-50 times. Microsatellite repeats are typically about 1-6 bases repeated up to six or more times. These repeats may occur many thousands of times throughout the genome. The nomenclature for tandem repeat loci is inexact. These and other tandem repeats may be referred to by the general, all-encompassing term variable numbers of tandem repeats, or VNTRs.

Genetic or forensic identification applications employing VNTRs can employ restriction fragment length polymorphism analysis (RFLP analysis), a gel-based method, or methods based on the polymerase chain reaction (PCR). RFLP analysis capitalizes on the differences in length between fragments of nucleic acids generated from non-compromised samples of nucleic acids by the use of restriction endonucleases. Restriction endonucleases, endonucleases for short, are enzymes that fragment, or cut, nucleic acids at highly predictable positions. If two intact samples of nucleic acids are cut by the same endonuclease, their fragment pattern will be identical if their genetic sequence is identical. If the samples are different, they will generate different fragments, based in part on the selection of cut sites at positions that will yield predictably different fragment sizes depending upon the occurrence of polymorphic tandem repeat loci within or at the cut site of a predicted fragment. Like many genetic or forensic identification applications employing tandem repeat loci, RFLP analysis relies upon the ability to separate, or resolve, the nucleic acid fragments based on their electrophoretic mobility through a sizing gel, or on other sizing protocols. Sizing-based protocols, however, are inherently limited by the resolving power of the sizing method; fragments that are either too small or differ only very slightly in size may not be resolvable. Although potentially a powerful genetic identification application, RFLP analysis generally requires fairly intact nucleic acid samples. Further, RFLP analysis requires considerable amounts of nucleic acids and requires a relatively long amount of time to generate and interpret results.

Genetic or forensic identification applications employing tandem repeat loci and PCR require less nucleic acids. In PCR-based applications, sequences containing loci with tandem repeat sequences are amplified, or copied, many times over and then typically separated and identified using sizing protocols. However, due to the nature of the PCR polymerase, and the nature of tandem repeat loci, PCR methods are prone to artifactual results due to “slippage,” or “stutter” during PCR amplification. Such slippage or stutter is due to the inability of the polymerizing enzyme to faithfully and accurately copy the sequences containing the tandem repeats. The nature of the tandem repeat sequence causes the PCR polymerase to sometimes skip and sometimes over-copy elements of the repeating units. As a result, the amplified copy of the sequence containing the tandem repeat is either longer or shorter than the original, thus failing to provide the fidelity required for genetic identification applications. Further, most PCR-based applications rely upon sizing methods for identification, and thus have the same drawbacks in this respect as does RFLP analysis. Due to the length of many useful tandem repeat loci, the amplified or copied sequences must be generally at least near a hundred and up to a thousand or more bases in length.

While these genetic or forensic identification techniques are very accurate and reliable, there may be spurious readings causing an inaccurate DNA profile, such as allele drop out, sample contamination causing foreign or extra alleles to appear in the DNA profile, or mixed profiles, PCR stutter and other conditions such as exposure of the sample to microorganisms, nuclease, or improper extraction of the sample that renders fewer than optimal number of intact useful loci available for genetic or forensic analysis. Errant interpretation of the data by the analyst can also occur.

Preparation of the biological controls used for genetic or forensic identification is often time consuming, subject to unintentional errors by laboratory personnel in making or using the control, such as, contamination of the sample all resulting in inaccurate, and unreliable results. In some instances, the quality and the integrity of the control cannot be relied upon. Thus, there is a need for compositions, methods and computerized systems that offer improved sensitivity, precision, reliability, and accuracy to biological assays.

### SUMMARY OF THE INVENTION

Compositions, methods and computerized systems are provided that improve sensitivity, precision, reliability, and accuracy to a biological assay.

In various embodiments, standardized biological controls are provided that reduce the time needed in preparing controls. These pre-made standardized controls prevent unintentional errors by laboratory personnel in making or using the control or running the assay.

In various embodiments, the biological controls of the present invention can be used to calibrate instruments and validate the processing protocols that are to be used in the biological assay.

In various embodiments, computerized systems are provided for performing human identity, validations of processes or instrumentation, proficiency testing, or training of laboratory personnel.

In one embodiment, a quality control kit for forensic testing of biological samples is provided, comprising: a container; and a support having at least one surface having a predetermined quantity of a biological sample from one or more known donors, wherein the biological sample is substantially free from contaminants.

In another embodiment, a quality control system for testing biological samples is provided, comprising: a support having at least one surface capable of receiving one or more predetermined quantities of a biological sample from one or more known donors.

In an exemplary embodiment, a system for quality control testing of biological samples is provided comprising: a) at least one quality control database for receiving and storing data associated with two or more different predetermined quantities of biological controls from one or more known donors; b) a processor for accessing and analyzing data from the at least one quality control database to assist in correlating a biological test result conducted by a user with the data associated with two or more different predetermined quantities of biological controls to determine if the user conducted the biological test properly; c) a storage device for storing the data analyzed by the processor; d) a user computer for making requests for quality control data to and for receiving quality control data from the processor; and e) a user interface for interfacing the processor and the user computer.

In another exemplary embodiment, a computer for managing quality control data from biological samples is provided comprising: a) at least one quality control database for storing data associated with two or more different predetermined quantities of biological controls from one or more known donors; b) a processor for accessing and analyzing data from the at least one quality control database to assist in correlating a biological test result conducted by a user with the data associated with two or more different predetermined quantities of biological controls to assist in determining if the user conducted the biological test properly, and c) a storage device for storing the data analyzed by the processor.

In yet another exemplary embodiment, a method of making quality control kits for testing of biological samples is provided comprising: providing a support having at least one surface capable of receiving one or more predetermined quantities of a biological sample from one or more known donors and filling the at least one surface of the support with the biological sample utilizing automated liquid handling robotics, cell sorters, fluorescence activated cell sorters, microtiter plate readers, real-time PCR DNA quantification systems or combinations thereof, thereby making the quality control kit.

Additional features and advantages of various embodiments will be set forth in part in the description that follows, and in part will be apparent from the description, or may be learned by practice of various embodiments. Other advantages of various embodiments will be realized and attained by means of the elements and combinations particularly pointed out in the description and appended claims.

### BRIEF DESCRIPTION OF THE FIGURES

FIG. 1 illustrates a block diagram of an embodiment of the system for quality control testing of biological samples.

FIG. 2 illustrates a block diagram of an embodiment of the system for quality control testing of biological samples.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION OF THE INVENTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a monomer” includes two or more monomers.

Reference will now be made in detail to certain embodiments of the invention. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

### Biological Sample

In various embodiments, the present invention comprises a quality control system for testing biological samples, comprising: a support having at least one surface capable of receiving one or more predetermined quantities of a biological sample from one or more known donors. The quality control system can be used to generate data from performance of the assay using the control and then compared to the data generated from the assay using the unknown sample. The data generated is compared during or after performance of the biological assay and the results compared to see the magnitude of variability.

In various embodiments, the support has at least two, three, four, five, six, or more surfaces capable of receiving one or more predetermined quantities of one or more the same or different predetermined quantities of a biological sample.

The quality control system of the present invention comprises a biological sample that is “substantially free from contaminants.” By “substantially free from contaminants” is meant that the sample is at least 90%, preferably at least 95% and, more preferably, at least 99% free of contaminants. Some examples of contaminants, include, non-biological material from a crime scene, contaminants from an organism other than the donor, lipids, carbohydrates, cellular debris or any material that substantially interferes with performing the present invention. The term “substantially free from contaminants” is not intended to refer to the absence of stabilizing agents such as water, buffers, or salts, unless they are present in amounts that substantially interfere with the methods of the present invention.

In various embodiments, the sample is essentially pure which means that the sample is free from materials used in the isolation and identification of the sample, such as, for example, affinity binding agents, separation agents, sodium dodecyl sulfate and other detergents.

Biological samples that can be used as controls in various embodiments of the present invention include, but are not limited to, any sample obtained from a living organism or an organism that was once alive including prokaryotic or eukaryotic cells. Examples of biological samples for use in the quality control system, include, but are not limited to, bacteria, virus, yeast, fungi, plant or animal cells. Suitable samples from animals include, but are not limited to, murine, human, ovine, equine, bovine, porcine, foul, canine or feline cells. Suitable bacterial cells include, but are not limited to, E. coli, Pseudomonas aeruginosa, Staphylococcus epidermidis, Staphylococcus aureus, Streptococcus pyogenes, Streptococcus pneumonia, or the like.

In various embodiments, the biological sample will be in the form of blood, vaginal fluid, semen, spermatozoa, saliva, oral epithelial cells, hair root cells, urine, teeth, bone, body tissue, or combination thereof from a known source or donor. By “known donor” or “known source” is meant the identity of the organism from which the sample is derived is known, or is known with a desired degree of at least 99%, for example 100% statistical certainty.

Known sources include, but is not limited to, sources where the genotype of the source is known. A “genotype,” as used herein, is meant the identities of STRs, SNPs, RFLPs, and/or nucleic acid sequence in the genomic sequence of the organism. In various embodiments, the known genotype was determined by mitochondrial DNA typing.

In various embodiments, the known genotype can be determined by using RFLP analysis. Typically, RFLP analysis uses restriction endonucleases, which cut the DNA at short, specific, internal nucleotide sequences, the fragments of which are usually 4-6 nucleotides in lengths. Once the fragments have been removed, they need to be separated to allow the comparison of the sizes of the DNA sequences. Electrophoresis exploits differences in size, shape and net charge of the molecules to separate them over an electric field. Following separation, the double-stranded DNA fragments are treated to separate the strands from each other and transferred to another support. Southern blotting is used to identify the DNA fragments. In Southern blotting the single stranded DNA is treated with radioactively labeled DNA containing a base sequence complementary to that of the fixed VNTRs. Any complementary bases will pair up and thus be bound to the support. Excess probe is washed away and the support is analyzed by autoradiography to show the labeled DNA as bands on the support. The length of each fragment is determined by running known DNA fragment lengths alongside the sample and comparing the distances migrated. When comparing the DNA fragment patterns of two or more samples, it is a match between the band sets that gives a positive result.

In various embodiments, the known genotype can be determined by STR analysis. Typically, STR analysis provides higher discriminating power than RFLP analysis but it also requires a smaller sample size. During forensic examination, an STR with a known repeat sequence is extracted and separated by electrophoresis often in the same way as RFLP analysis. By examining the distance the STR has migrated via electrophoresis on the support, the number of STRs can be determined. There are hundreds of types of STRs and the more STRs that can be characterized, the less chance there is of the DNA of two individuals giving the same results. The effectiveness of STR technique has lead to multiplexing, the extraction and analysis of a combination of different STRs. Combining the technology of PCR with STR analysis enables the simultaneous extraction and amplification of the DNA fragments. The selection of STRs ensures complete separation and clarity of results and can also include the use of fluorescent dyes to visualize the STR loci. Separation can also be accomplished by capillary electrophoresis as known in the art.

In various embodiments, the known genotype can be determined by mitochondrial DNA typing. Mitochondrial DNA typing is a method known in the art. Typically, Mitochondrial DNA provides another way for forensic DNA typing or profiling. For example, the high number of sequence variants in the two hypervariable portions of the non-coding control region of the human mtDNA molecule allows discrimination among individuals and/or biological samples. Mitochondrial DNA is inherited from the mother only, so that in situations where an individual is not available for a direct comparison with a biological sample, any maternally related individual may provide a reference sample. An mtDNA analysis, typically, begins with the extraction of, for example, total genomic DNA from an unknown biological sample, such as a tooth, blood sample, or hair. PCR is then used to amplify the two hypervariable portions of the non-coding region of the mtDNA molecule, using flanking primers. When adequate amounts of PCR product are amplified to provide the necessary information about the two hypervariable regions the sequences of both hypervariable regions are determined on both strands of the double-stranded DNA molecule, with sufficient redundancy to confirm the nucleotide substitutions that characterize that particular sample. The entire process is then repeated with a known sample, such as blood or saliva collected from a known individual. The sequences from the known samples and unknown sample are compared to determine if they match.

When the biological control comprises materials such as DNA, RNA, DNA/RNA hybrids, mitochondrial DNA (mtDNA), proteins, amino acids, or cells. Such material may be obtained by extraction methods known to those of ordinary skill in the art, for example, organic or liquid extraction, solid phase extraction, magnetic bead separation, centrifugation or the like.

In various embodiments, the DNA may be amplified to the appropriate quantity using methods known in the art such as the polymerase chain reaction. This quantity of DNA can be used as the control for the biological assay.

In various embodiments, if the sample is DNA, the DNA may contain STRs, minisatellite repeats, or Y-STRs (STRs along the Y-chromosome), VNTRs, SNPs that will generate a known DNA profile, for example, on an electropherogram. In various embodiments, the DNA comprises mitochondrial DNA.

Typical STRs, SNPs, mtDNA or RFLP, used in forensic are known to those of ordinary skill in the art. STRs database is available over the Internet at http://www.cst1.nist.gov/div831/strbase/index.htm, and 13 CODIS Core STR Loci with chromosomal positions are described at http://www.cst1.nist.gov/div831/strbase/fbicore.htm. For a review of the methods of forensic identification, see for example, Jobling and Gill Encoded Evidence: DNA in Forensic Analysis, Nature Reviews Genetics 5, 739-751 (2004). An example of a known database for mtDNA is the SWGDAM mtDNA database.

Typically, in forensic identification, PCR-based STRs are preferred over conventional Southern blotting techniques of the larger variable number tandem repeats (VNTRs). Discrete alleles from STR systems may be obtained due to their smaller size, which puts them in the size range where DNA fragments differing by a single base pair in size may be differentiated. In addition, smaller quantities of DNA, including degraded DNA, may be typed using STRs.

In various embodiments, the STRs, minisatellite repeats, VNTRs, mtDNA, SNPs are provided as panels from known donors.

When the biological control comprises DNA, in various embodiments, the DNA may contain known polymorphisms or mutations in the form of deletions, insertions, re-arrangement, repetitive sequence, base modifications, or single or multiple base changes at a particular site in a nucleic acid sequence. Such mutations or polymorphisms include, but are not limited to, single nucleotide polymorphisms (SNPs), one or more base deletions, or one or more base insertions.

One preferred method of detecting polymorphic sites employs enzyme-assisted primer extension. SNP-IT™ (disclosed by Goelet, P. et al., and U.S. Pat. Nos. 5,888,819 and 6,004,744, each herein incorporated by reference in its entirety) is a preferred method for determining the identity of a nucleotide at a predetermined polymorphic site in a target nucleic acid sequence. Thus, it is uniquely suited for SNP scoring, although it also has general applicability for determination of a wide variety of polymorphisms. SNP-IT™ is a method of polymorphic site interrogation in which the nucleotide sequence information surrounding a polymorphic site in a target nucleic acid sequence is used to design an oligonucleotide primer that is complementary to a region immediately adjacent to, but not including, the variable nucleotide(s) in the polymorphic site of the target polynucleotide. The target polynucleotide is isolated from a biological sample and hybridized to the interrogating primer. Following isolation, the target polynucleotide may be amplified by any suitable means prior to hybridization to the interrogating primer. The primer is extended by a single labeled terminator nucleotide, such as a dideoxynucleotide, using a polymerase, often in the presence of one or more chain terminating nucleoside triphosphate precursors (or suitable analogs). A detectable signal is thereby produced. As used herein, immediately adjacent to the polymorphic site includes from about 1 to about 100 nucleotides, more preferably from about 1 to about 25 nucleotides in the 5′ direction of the polymorphic site, with respect to the directionality of the target nucleic acid. Most preferably, the primer is hybridized one nucleotide immediately adjacent to the polymorphic site in the 5′ direction with respect to the polymorphic site.

In some embodiments of SNP-IT™, the primer is bound to a solid support prior to the extension reaction. In other embodiments, the extension reaction is performed in solution (such as in a test tube or a microwell) and the extended product is subsequently bound to a solid support. In an alternate embodiment of SNP-ITT, the primer is detectably labeled and the extended terminator nucleotide is modified so as to enable the extended primer product to be bound to a solid support. An example of this includes where the primer is fluorescently labeled and the terminator nucleotide is a biotin-labeled terminator nucleotide and the solid support is coated or derivatized with avidin or streptavidin. In such embodiments, an extended primer would thus be capable of binding to a solid support and non-extended primers would be unable to bind to the support, thereby producing a detectable signal dependent upon a successful extension reaction.

An alternate method for determining the identity of a nucleotide at a polymorphic site in a target polynucleotide is described in Söderlund et al, U.S. Pat. No. 6,013,431 (the entire disclosure of which is herein incorporated by reference). In this method, the nucleotide sequence surrounding a polymorphic site in a target nucleic acid sequence is used to design an oligonucleotide primer that is complementary to a region flanking the 5′ end, with respect to the polymorphic site, of the target polynucleotide, but not including the variable nucleotide(s) in the polymorphic site of the target polynucleotide. The target polynucleotide is isolated from the biological sample and hybridized with an interrogating primer. In some embodiments of this method, following isolation, the target polynucleotide may be amplified by any suitable means prior to hybridization with the interrogating primer. The primer is extended, using a polymerase, often in the presence of a mixture of at least one labeled deoxynucleotide and one or more chain terminating nucleoside triphosphate precursors (or suitable analogs). A detectable signal is produced on the primer upon incorporation of the labeled deoxynucleotide into the primer.

In various embodiments, the control is used for determining at least: (i) the likelihood that an individual is or is not the progeny of the putative male or female parent or grandparent, (ii) the likelihood that an individual committed a crime, or (iii) the likelihood that an individual is an ancestor or relative.

### Predetermined Quantities

The biological sample whether DNA, RNA, DNA/RNA hybrids, mitochondrial DNA, proteins, amino acids, or cells can be provided in “predetermined quantities”. By predetermined quantity is meant that the biological sample is provided in a known amount, for example, concentration, mass, volume or combination thereof. These biological samples will be distributed as the quality control product, saving the laboratory time in preparing biological controls, avoid unintentional errors by laboratory personnel in making or using the control or running the assay. The biological controls of the present invention can be used to calibrate instruments, validate protocols, serve as proficiency tests, and the like that are to be used in the biological assay. They may also. serve as training aids and compose inferences that the experimental data collected for questions samples co-processed with biological control samples have generated correct typings.

Concentrations of the biological sample can be provided typically in aliquots of increasing quantities and sent to the laboratory to be used as the control. It is envisioned that these biological samples will be approved and standardized by NIST, FDA, NIH, CDC, ASTM or other agency.

In various embodiments, the biological sample comprises cells from a known donor. Quantities of cells can be in increasing aliquots from, for example, 1 cell to 1×10⁸ cells or more. These cells will be provided on supports in increasing quantities, such as for example, 1×10¹, 1×10²,1×10³, 1×10⁴ cells, etc. The cells can be, for example, male or female cells or combinations thereof.

In various embodiments, the cells are provided in suitable media to maintain the integrity of the cells. Different cell types often require the use of different media formulations. Typical components of cell media include amino acids, organic and inorganic salts, stabilizers, diluents, buffers, vitamins, trace metals, sugars, lipids and nucleic acids, the types and amounts of which may vary depending upon the particular requirements of a given cell or tissue type.

In various embodiments, the biological sample comprises DNA from a known donor. Quantities of DNA can be in increasing aliquots from, for example, 1 picogram to 1 microgram or higher. The DNA will be provided in increasing quantities, such as for example, 3 picogram, 6 picograms, 9 picograms, etc. Typically, the DNA will be provided in ready quantities needed to perform the biological assay in suitable media such as salts, stabilizers, diluents, buffers, etc.

### Supports

In various embodiments, the biological sample is provided in a support having at least one surface capable of receiving one or more predetermined quantities of a biological sample from one or more known donors. The support can be any material capable of holding the biological sample. Suitable supports comprise nitrocellulose, cellulose, paper, silica gel, silicon, glass, polystyrene, nylon, polypropylene, CPG or combinations thereof. The biological sample can be provided in array format, where more than one sample in the same or different quantities can be placed in discrete positions of the support. In various embodiments, different samples may be placed in the array, for example, a predetermined quantity of DNA containing STRs may be placed in one surface on the array, while DNA containing SNPs may be placed in another surface on the array. Thus, it is contemplated that multiplex assays can be conducted or run in parallel.

In various embodiments, it is contemplated that the support comprises filter paper impregnated with DNA or cells of different quantities. In various embodiments, the support comprises a column with DNA or cells of different quantities.

In various embodiments, the support may contain a plurality of wells for holding the sample and be arranged in, for example, format such as those found on 96-well plates (12 times 8 array of wells), 384-well plates (24 times 16 array of wells) and 1536-well plate (48 times 32 array of well). However, unlike conventional microtiter plates, in various embodiments of the present invention, predetermined quantities of the biological sample (that is substantially free-from contaminants) of one or more known donors is disposed in each well of the plate in increasing aliquots.

In various embodiments, the solid support has more than one surface of different sizes to hold different quantities of the biological specimen. For example, the support may contain at least two, three, four, five, six or more surfaces or wells that can hold increasing quantities of a sample, such as for example, 0.05 microliters of a sample, while a different well in the same support can hold 0.1 microliters, while a different well in the same support can hold 0.1 microliters, etc. The support, for example, may have different size surfaces that hold different quantities of the known biological sample.

In various embodiments of the present invention, the biological sample is disposed or filled on at least one surface of the support. Many methods are available to dispose biological samples in discrete areas of a support. Some methods include, but are not limited to, automated liquid handling robotics, cell sorters, fluorescence activated cell sorters, microtiter plate readers, and real-time PCR DNA quantification systems. In various embodiments pulse-jet techniques can be used. Pulse-jet provides uniform and reproducible deposition of biological sample on supports. Pulse-jet is a non-contact technique, pulse-jet deposition does not result in scratching or damaging the surface of the support on which the biological sample is deposited.

In various embodiments, kits are provided comprising a container; and a support having at least one surface having a predetermined quantity of a biological sample from one or more known donors, wherein the biological sample is substantially free from contaminants. The kit may also contain a negative control as well as a positive control in addition to reagent(s) and/or active and/or inert ingredient(s) for performing the biological assay. In various embodiments, the kit may contain instructions for mixing or combining ingredients or use. The kits can be safely wrapped for transport of biological samples and sealed in a container, for example, a tamper resistant container to further ensure integrity of the biological control.

### System

In various embodiments, the quality controls of the present invention can be adapted for access via a computer interface. In various embodiments, the quality control system comprises at least one quality control database for receiving and storing data associated with at least one predetermined quantity of a biological control from one or more known donors. This quality control database can be maintained on a server and updated with new biological sample controls. The quality control database contains data in computer readable form of the biological control, e.g., image or information of the DNA profile or image from optically scanned or read microtiter plates. In various embodiments, the quality control database can be linked to the CODIS database or the SWGDAM mtDNA database and be available for access by law enforcement.

In various embodiments, the server includes a processor, quality control storage database and a user interface. The processor, quality control database and quality control storage database and user interface may, optionally, reside on one server.

The quality control database can be accessed by the processor in order to access and analyze data from the quality control database to assist in correlating a biological test result conducted by a user with the data associated from one or more predetermined quantities of the biological control to determine if the user conducted the biological test properly. Comparison or calculation software to compare or calculate two or more different data sets is known in the art. The quality control storage database can store the data analyzed that a user can access. An e-mail can be generated to the user containing biological test information.

In various embodiments, the quality control system of the present invention also comprises a user computer for making requests to and receiving information from the quality control database, and/or the processor.

In various embodiments, the user computer comprises a user interface for interfacing the processor and the user computer. The user interface may include software to access the quality control system.

In various embodiments of the present invention, the processor, the quality control data storage device and the user interface reside on a server.

In various embodiments, the user computer requires a security password or encryption code before interfacing with the processor, wherein the interfacing is conducted using a web browser.

In various embodiments of the present invention, it is contemplated that the computerized system can be used for proficiency testing, quality assessment, assay validation, training of technicians, product acquisitions, results verification, blind quality assessment, and/or laboratory certification. It is contemplated that the system can be used in laboratories, such as for example, local, state, international crime labs, forensic labs and by law enforcement agencies.

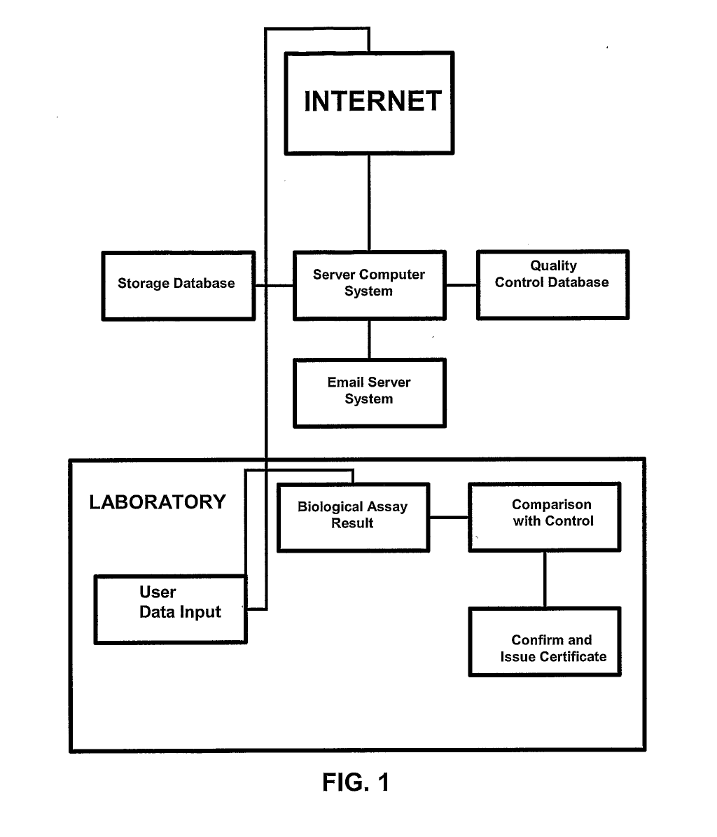

FIG. 1 illustrates a block diagram of a preferred embodiment of the system for quality control testing of biological samples. The user laboratory conducting the biological assay accesses the system via the Internet, a password or code is needed to access the biological control server system, which is verified and allows the user access to the system. The biological control server system allows the user to input the barcode for the biological assay or data from the biological assay, for example, from an STR electropherogram. The quality control database allows the user access to information on the biological control, such as for example, the electropherogram for the biological control, which may be stored in the storage database. The control and the unknown sample can be compared for matches by the processor and the peaks analyzed to determine if the experiment was run properly. A certification can be sent to the user, such as for example, by e-mail from the server computer or a certificate can be printed recording the transaction. The history of this transaction can be stored on the storage database. Once the results are obtained from the assay, a storage device will record the data in computer readable form. This data can be archived or retrieved at another date from the storage database to show histories of, for example, the overall performance of the laboratory personnel performing the assay.

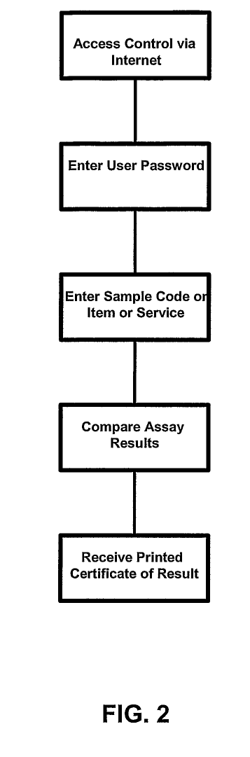

FIG. 2 illustrates a block diagram of a preferred embodiment of the system from the user laboratory's computer. The user via the user interface computer will access the biological control database by inputting a password or bar code associated with the biological control into the system or an item or service to be selected, such services can be to order more control or provide proficiency testing to the user, or to obtain information on the biological control. The assay result is compared to the control by the processor, then the comparison result is sent to the user computer and if appropriate a printed certificate is generated from the user's computer.

In various embodiments, the invention provides a method of making quality control kits for testing of biological samples comprising providing a support having at least one surface capable of receiving one or more predetermined quantities of a biological sample from one or more known donors and filling the at least one surface of the support with the biological sample utilizing automated liquid handling robotics, cell sorters, fluorescence activated cell sorters, microtiter plate readers, real-time PCR DNA quantification systems or combinations thereof, thereby making the quality control kit.

In various embodiments control kits are provided, wherein the kits are used for determining at least: (i) the likelihood that an individual is or is not the progeny of the putative male or female parent or grandparent, (ii) the likelihood that an individual committed a crime, or (iii) the likelihood that an individual is an ancestor or relative. The biological samples of the kits can be from bacteria, viruses, fungi, plants, or animals. In various embodiments, the animal is murine, human, ovine, equine, bovine, porcine, fowl, canine, or feline.

Having now generally described the invention, the same may be more readily understood through the following reference to the following examples, which are provided by way of illustration and are not intended to limit the present invention unless specified.

### EXAMPLES

### Example 1

Table 1 shows DNA profiles of STRs that includes the 13 CODIS STR loci D3S1358 (D3), VWA, FGA, D8S1179 (D8), D21S11 (D21), D18S51 (D18), D5S818 (D5), D13S317 (D13), D7S820 (D17), D16S539 (D16), THO1, TPOX, CSF1PO (CSF), AMEL, and D2S1338 (D2), and D19S4×(D19). These loci are known in to those of ordinary skill in the art and have GenBank Accession number listed in Table A below.

TABLE A
GENBANK ACCESSION NUMBERS OF STR LOCI

| STR LOCUS NAME | GENBANK ACCESSION NO. |
|---|---|
| TPOX | M68651 |
| D2S1338 (D2) | G08202 |
| D19S433 (D19) | G08036 |
| D3S1358 (D3) | 11449919 |
| vWA | M25858 |
| FGA | M64982 |
| D8S1179 (D8) | G08710 |
| D21S11 (D21) | M84567 |
| D18S51 (D18) | L18333 |
| D5S818 (D5) | G08446 |
| D13S317 (D13) | G09017 |
| D7S820 (D17) | G08616 |
| D16S539 (D16) | G07925 |
| THO1 | D00269 |
| CSF1PO (CSF) | X14720 |
| AMELOGENIN | M55418 & M55419 |

The sample was from a donor that had unknown genotypes and was obtained as a buffy coat from blood. The genotype for each allele is indicated. DNA was extracted using FTA and stain extraction buffer protocols (SEB).

SEB Extraction Protocol. 1⅛ mm punch of S&S and FTA card was placed in a 1.7 ml centrifuge tube. An entire swab was placed in a similar tube. The hole puncher was cleaned between samples by punching 4 holes in clean Whatman paper. Fifty microliters of Proteinase K (10 mg/ml) were added, followed by 600 μl of SEB buffer. The tubes were vortexed and incubated at 65° C. for one hour. Six hundred microliters of Phenol:Chloroform were added, and the tubes were vortexed and centrifuged at 14,000 rpm for 20 minutes. About 500 μl of the top layer was transferred into new 1.7 ml centrifuge tubes containing 800 μl of 95% ethanol and 50 μl of sodium acetate. The samples were inverted ten times and centrifuged for 20 minutes at 14,000 rpm. The supernatant was poured off and 600 μl of 70% ethanol was added, and the samples were centrifuged at 4,000 rpm for 10 minutes, after which the alcohol was poured off, the tubes blotted, and the samples dried at 45° C. for 30 minutes. Twenty microliters of sterile water was added and the samples vortexed briefly. The samples were then incubated at 65° C. for 5 minutes. The samples were then vortexed briefly and incubated at 65° C. for another 5 minutes.

FTA Extraction Protocol. In the first step, 1⅛ mm punch was aliquoted into a 1.5 ml centrifuge tube by the same process as described above. In the second step, 500 μl of FTA Purification Reagent (Cat.# WB120204, Lot# 3101052) was added, and the tube was incubated at room temperature for 1 minute, then a pipet was used to remove all the reagent. In the third step, the second step was repeated for a total of three washes. In the fourth step, 495 μl of FTA Purification Reagent and 5 μl of Proteinase K (10 mg/ml) were then added. The tube was incubated at 65° C. for 1 hour. In the fifth step, the contents were transferred to a 2 ml centrifuge tube with a basket and centrifuged at 6,000×g for 30 seconds. In the sixth step, the basket was removed and the reagent discarded, and 500 μl of TE-1 (Teknova Cat.# T0223, Lot #T022314E401) was added and the tube was centrifuged at 6,000×g for 30 seconds. In the seventh step, the sixth step was repeated for a total of 3 washes. The punch was then placed in a 96-well PCR plate for amplification.

All the samples were amplified using Applied Biosystems AmpF1STR Identifiler PCR Amplification Kit (Part Number 4322288). The amplifications were carried out according to the AmpF1 lSTR Identifiler PCR amplification Kit User's Manual. The samples were analyzed on an ABI Prism3100 Genetic Analyzer and were loaded according to the User's Manual. The genotypes were generated using the ABI Prism Data Collection Software.

Table 1 shows for cell counts of 50, 100, 200, 300 and 464 on FT paper, as the cell count increases the genotype for STR loci can be determined on an increasing number of loci and in some cases a full profile is generated. Genotypes for samples extracted from FTA paper using the SEB extraction process could not be determined at the described cell count.

TABLE 1
Sample Extracted with FTA Protocol

| Sample ID | D8 | D21 | D7 | CSF | D3 | THO1 | D13 | D16 |
|---|---|---|---|---|---|---|---|---|
| FTA-50 | | | | | | | | |
| FTA-100 | 15 | | | | 13, 18 | 6 | 11 | |
| FTA-200 | 13, 15 | 32, 32.2 | | 11 | 13, 18 | 6, 9.3 | 11 | |
| FTA-300 | 13, 15 | 32, 32.2 | 10, 12 | 10, 11 | 13, 18 | 6, 9.3 | 11 | 11, 14 |
| FTA-464 | 13, 15 | 32, 32.2 | 10, 12 | 10, 11 | 13, 18 | 6, 9.3 | 11 | 11, 14 |
| FTSE50 | | | | | | | | |
| FTSE100 | | | | | | | | |
| FTSE200 | | | | | | | | |
| FTSE300 | | | | | | | | |
| FTSE464 | | | | | | | | |
| SS50 | | | | | | | | |
| SS100 | | | | | | | | |
| SS200 | | | | | | | | |
| SS300 | | | | | | | | |
| SS464 | | | | | | | | |
| SWAB50 | | | | | | | | |
| SWAB100 | 15 | | | | | 9 | | |
| SWAB200 | | | | | | | | |
| SWAB300 | | | | | | | | |
| SWAB464 | | | | | | | | |

| | | | | | | | | |
|---|---|---|---|---|---|---|---|---|
| Sample ID | D2 | D19 | vWA | TPOX | D18 | AMEL | D5 | FGA |
| FTA-50 | | | | | | | | |
| FTA-100 | | 14 | 15 | | | X | 11 | 22 |
| FTA-200 | 20 | 14, 15 | 15, 20 | | | X | 11 | 22 |
| FTA-300 | 17, 20 | 14, 15 | 15, 20 | 8, 11 | 18, 20 | X | 11 | 22 |
| FTA-464 | 17, 20 | 14, 15 | 15, 20 | 8, 11 | 18, 20 | X | 11 | 22 |
| FTSE50 | | | | | | | | |
| FTSE100 | | | | | | | | |
| FTSE200 | | | | | | | | |
| FTSE300 | | | | | | | | |
| FTSE464 | | | | | | | | |
| SS50 | | | | | | | | |
| SS100 | | | | | | | | |
| SS200 | | | | | | | | |
| SS300 | | | | | | | | |
| SS464 | | | | | | | | |
| SWAB50 | | | | | | | | |
| SWAB100 | | | | 8 | | X | | |
| SWAB200 | | | | | | | | |
| SWAB300 | | | | | | X | 11 | |
| SWAB464 | | | | 8 | | X | | |

table 1 legend:
FTA: FTA paper with FTA extraction;
FTSE: FTA paper with SEB extraction;
SS: S & S paper with SEB extraction;
SWAB; Cotton swab with SEB extraction;
* All cells obtained from Carter Blood Care Buffy Coat.

### Example 2

Table 2 shows DNA profiles of STRs that includes the 13 CODIS STR loci D3S1358 (D3), VWA, FGA, D8S1179 (D8), D21S11 (D21), D18S51 (D18), D5S818 (D5), D13S317 (D13), D7S820 (D17), D16S539 (D16), THO1, TPOX, CSF1PO (CSF), AMEL, and D2S1338 (D2), and D19S433 (D19). The sample used consisted of mononuclear cells (which include white blood cells) obtained from bone marrow of an unknown donor and was obtained from Cambrex Bio Science Walkersville, Inc. The genotype for each allele is indicated. DNA was extracted using FTA, FASTRACT, and CHELEX extraction protocols.

FASTRACT Extraction Protocol. 1⅛ mm punch of each card was placed into a 1.7 ml centrifuge tube; alternatively, one swab was placed into a 2.0 ml centrifuge tube. FASTRACT solution (200 μl) was added, and the tube vortexed. The tube was incubated at 56° C. for 15 minutes, then at 94° C. for 10 minutes. The tubes were then vortexed and the samples centrifuged.

Chelex Extraction Protocol. In the first step, either one or two punches, depending on the amount of cells needed, were aliquoted into a 1.7 ml tube. The hole puncher was cleaned by punching four times onto clean Whatman paper. In the second step, 400 μl of TE (pH 8.0) was added to each sample, and the sample incubated at room temperature for 10 minutes. In the third step, a P1000 pipettor was used to remove the TE from each tube, ensuring that the punch remained in the tube. In the fourth step, the second and third steps were repeated for a total of three washes. During the 10 minute incubations, a 5% Chelex solution (Chelex 100) was prepared by pouring 20 ml of sterile DI water into a clean 50 ml beaker with a stir bar. The beaker was placed on a stir plate, and 1 gram of Chelex beads was added to the water in the beaker, and the mixture stirred until the beads were completely suspended. The stir plate was turned off, and the beads allowed to settle. Ten to 15 ml of water were removed and replaced with an equal volume of DI water, and the suspension stirred again to resuspend the beads, with the suspension stirring while dispensing it. The Chelex was dispensed using a combitip with its end cut off. Two hundred microliters of Chelex was added to the tubes, inverting the repeater between samples to prevent the beads from settling. The tubes were closed, vortexed, briefly spun, and incubated at 56° C. for 15 minutes in a water bath. After 15 minutes, the tubes were removed from the water bath and vortexed, then incubated at 95° C. for 8 minutes. The tubes were then removed from the water bath, vortexed, and briefly spun. The samples were stored at minus 20° C. until ready for use.

FTA Extraction ⅛ mm punch. In the first step, a 1⅛ mm punch was aliquoted into the basket of a 2.0 ml centrifuge tube by the same process as above. In the second step, 500 μl of FTA Purification Reagent (Cat.# WB120204, Lot# 3101052) was added to the tube, and the tube was incubated at room temperature for 1 minute, after which the tube was centrifuged at 6000×g for 30 seconds. In the third step, the basket was removed and the used reagent was decanted. In the fourth step, the tube was returned to the basket and the second and third steps were repeated for a total of three washes. In a fifth step, the punch was moved from the basket to the 2.0 ml tube using a clean pipet tip. In a sixth step, 495 μl of FTA Purification Reagent and 5 μl of ProK (10 mg/ml) were added, and incubation at 65° C. for 1 hour was carried out. In a seventh step, the contents were transferred to the basket of a 2.0 ml centrifuge tube and centrifuges at 6,000×g for 30 seconds. In the eighth step, the basket was removed and the reagent discarded, and 500 μl of TE⁻¹ (Teknova Cat.# T0223, Lot #T022314E401) was added and centrifugation was carried out at 6,000×g for 30 seconds. In the ninth step, the sixth step was then repeated for a total of three washes. Finally, the punch was placed in 96-well PCR plate for amplification.

FTA Extraction 16 mm punch. In the first step, a 1-16 mm punch into the basket of a 2.0 ml centrifuge tube by the same process as above, ensuring that the punch fitted securely on the bottom of the basket. In the second step, 500 μl of FTA Purification Reagent (Cat.# WB120204, Lot# 3101052) was added, and incubation was carried out at room temperature for 1 minute, and the tube centrifuged at 6,000×g for 30 seconds. In the third step, the basket was removed and the used reagent decanted. In the fourth step, the basket was returned to the tube and the second and third step were repeated for a total of three washes. In the fifth step, 5 μl of ProK (10 mg/ml) and 495 μl of FTA Purification Reagent were added and an incubation at 65° C. was carried out for 1 hour. In the sixth step, centrifugation was carried out at 6,000×g for 30 seconds. Finally, the basket was removed, the reagent discarded, 500 μl of TE⁻¹ (Teknova Cat.# T0223, Lot #T022314E401) was added, and centrifugation was carried out at 6,000×g for 30 seconds.

Table 2 shows as the cell count increases the genotype for STR loci can be determined on an increasing number of loci and in some cases a full profile is generated. Results for ⅛ inch punches were superior to the results obtained from 16 mm punches. Genotypes for samples extracted from FTA paper using the FASTRACT extraction process could not be determined.

TABLE 2

| | | | | | | | | | | | | | | | | | | | | Systems |
|---|---|---|---|---|---|---|---|---|---|---|---|---|---|---|---|---|---|---|---|---|
| | Cell | | | | | | | | | | | | | | | | | | | Genotyped |
| Sample ID | Count | Paper | Extraction | D19 | VWA | TPOX | D18 | AME | D5 | FGA | D8 | D21 | D7 | CSF | D3 | TH01 | D13 | D16 | D2 | Correctly |
| 50CF1 | 50 | FTA | Fastract | | | | | | | | | | | | | | | | | 0 |
| 100CF1 | 100 | FTA | Fastract | | | | | | | | | | | | | | | | | 0 |
| 200CF1 | 200 | FTA | Fastract | | | | | | | | | | | | | | | | | 0 |
| 300CF1 | 300 | FTA | Fastract | | | | | | | | | | | | | | | | | 0 |
| 430CF1 | 430 | FTA | Fastract | | | | | | | | | | | | | | | | | 0 |
| 430X2CF1 | 860 | FTA | Fastract | | | | | | | | | | | | | | | | | 0 |
| 50CF2 | 50 | FTA | Chelex | | | | | | | | | | | | | | | | | 0 |
| 100CF2 | 100 | FTA | Chelex | | | | | | | | | | | | | | | | | 0 |
| 200CF2 | 200 | FTA | Chelex | | | | | | | | | | | | | | | | | 0 |
| 300CF2 | 300 | FTA | Chelex | 14 | | | | X | | | 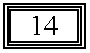 | | | | | 6 | | | | 3 |
| 430CF2 | 430 | FTA | Chelex | 14 | | | | X | 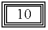 | | | | | | | | | | | 2 |
| 430X2CF2 | 860 | FTA | Chelex | 14 | 16, 17 | 8, 11 | | X | 10, 11 | | 12, 14 | | | | 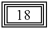 | 6 | 11, 13 | 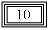 | 17, 25 | 9 |
| 50CF3 | 50 | FTA | FTA-1/8punch | 14 | 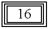 | 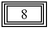 | | X | 10, 11 | | 12, 14 | 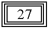 | | 12 | 15, 18 | 6 | 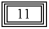 | | | |
| 100CF3 | 100 | FTA | FTA-1/8punch | 14 | 16, 17 | | 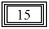 | X | 10, 11 | | 12, 14 | | | 12 | 15, 18 | 6 | 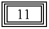 | | | 8 |
| 200CF3 | 200 | FTA | FTA-1/8punch | 14 | 16, 17 | 8, 11 | 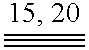 | X | 10, 11 | 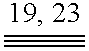 | 12, 14 | 27, 31.2 | 10, 11 | 12 | 15, 18 | 6 | 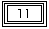 | 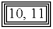 | 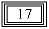 | 14 |
| 300CF3 | 300 | FTA | FTA-1/8punch | 14 | 16, 17 | 8, 11 | 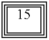 | X | 10, 11 | 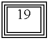 | 12, 14 | 27, 31.2 | 10, 11 | 12 | 15, 18 | 6 | 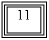 | 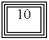 | 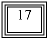 | 11 |
| 430CF3 | 430 | FTA | FTA-1/8punch | 14 | 16, 17 | 8, 11 | 15, 20 | X | 10, 11 | 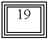 | 12, 14 | 27, 31.2 | 10, 11 | 12 | 15, 18 | 6 | 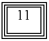 | 10, 11 | 17, 25 | 14 |
| 430X2CF3 | 860 | FTA | FTA-1/8 | 14 | | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 15 |
| | | | punch | | | | | | | | | | | | | | | | | |
| 50CF4 | 50 | FTA | FTA-16 mm | | | | | | | | | | | | | | | | | 0 |
| | | | punch | | | | | | | | | | | | | | | | | |
| 100CF4 | 100 | FTA | FTA-16 mm | | | | | | | | | | | | | | | | | 0 |
| | | | punch | | | | | | | | | | | | | | | | | |
| 200CF4 | 200 | FTA | FTA-16 mm | | | | | | | | | | | | | | | | | 0 |
| | | | punch | | | | | | | | | | | | | | | | | |
| 300CF4 | 300 | FTA | FTA-16 mmpunch | 14 | | | | X | | | 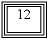 | | | | | | | | | 2 |
| 430CF4 | 430 | FTA | FTA-16 mm | | | | | | | | | | | | | | | | | 0 |
| | | | punch | | | | | | | | | | | | | | | | | |
| 860CF4 | 860 | FTA | FTA-16 mmpunch | 14 | 16, 17 | 8, 11 | 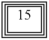 | X | 10, 11 | | 12, 14 | 27, 31.2 | | 12 | 15, 18 | 6 | | 10, 11 | 17, 25 | 12 |
| 50C51 | 50 | S&S | Fastract | 14 | 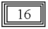 | | | | | | | | | | | 6 | 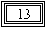 | | | 2 |
| 100CS1 | 100 | S&S | Fastract | 14 | | | | X | 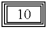 | | | | 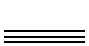 | | | 6 | | | | 3 |
| 200CS1 | 200 | S&S | Fastract | 14 | 16, 17 | 8, 11 | 15, 20 | X | 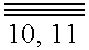 | 19, 23 | 12, 14 | 27, 31.2 | 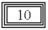 | 12 | 15, 18 | 6 | 11, 13 | 10, 11 | 17, 25 | 15 |
| 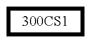 | 300 | S&S | Fastract | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 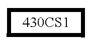 | 430 | S&S | Fastract | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 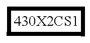 | 860 | S&S | Fastract | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 50CS2 | 50 | S&S | Chelex | | | | | | | | | | | | | | | | | 0 |
| 100CS2 | 100 | S&S | Chelex | | | | | | | | | | | | | 6 | | | | 1 |
| 200CS2 | 200 | S&S | Chelex | 14 | 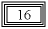 | | | X | | | | | | | | | | | | 2 |
| 300CS2 | 300 | S&S | Chelex | 14 | | 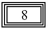 | | X | 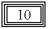 | | | | | | | 6 | | | | 2 |
| 430CS2 | 430 | S&S | Chelex | 14 | 16, 17 | 8, 11 | | X | 10, 11 | | 12, 14 | 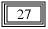 | | 12 | 15, 18 | 6 | 11, 13 | 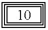 | 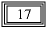 | 10 |
| 430X2CS2 | 860 | S&S | Chelex | 14 | 16, 17 | 8, 11 | 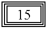 | X | 10, 11 | | 12, 14 | 27, 31.2 | | 12 | 15, 18 | 6 | 11, 13 | 10, 11 | 17, 25 | 13 |
| 50CS3 | 50 | S&S | FTA-1/8 | 14 | | | | | 10, 11 | | | | | | | | | | | 2 |
| | | | punch | | | | | | | | | | | | | | | | | |
| 100CS3 | 100 | S&S | FTA-1/8punch | 14 | 16, 17 | 8, 11 | | X | 10, 11 | 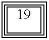 | 12, 14 | 27, 31.2 | | 12 | 15, 18 | 6 | 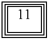 | 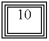 | 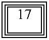 | 10 |
| 200CS3 | 200 | S&S | FTA-1/8punch | 14 | 16, 17 | 8, 11 | 15, 20 | X | 10, 11 | 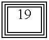 | 12, 14 | 27, 31.2 | 10, 11 | 12 | 15, 18 | 6 | 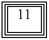 | 10, 11 | 17, 25 | 14 |
| 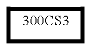 | 300 | S&S | FTA-1/8punch | 14 | 16, 17 | 8, 11 | 15, 20 | X | 10, 11 | 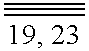 | 12, 14 | 27, 31.2 | 10, 11 | 12 | 15, 18 | 6 | 11 | 10, 11 | 17, 25 | 16 |
| 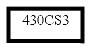 | 430 | S&S | FTA-1/8punch | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 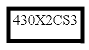 | 860 | S&S | FTA-1/8punch | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 50CS4 | 50 | S&S | FTA-16 mm | | | | | | | | | | | | | | | | | 0 |
| | | | punch | | | | | | | | | | | | | | | | | |
| 100CS4 | 100 | S&S | FTA-16 mm | | | | | | | | | | | | | | | | | 0 |
| | | | punch | | | | | | | | | | | | | | | | | |
| 200CS4 | 200 | S&S | FTA-16 mm | | | | | | | | | | | | | | | | | 0 |
| | | | punch | | | | | | | | | | | | | | | | | |
| 300CS4 | 300 | S&S | FTA-16 mm | | | 8, 11 | | X | | | 12, 14 | | | | | | | | | 3 |
| | | | punch | | | | | | | | | | | | | | | | | |
| 430CS4 | 430 | S&S | FTA-16 mm | 14 | 16, 17 | 8, 11 | 15, 20 | X | 10, 11 | | 12, 14 | 27, 31.2 | | 12 | | 6 | | 10, 11 | 17, 25 | 12 |
| | | | punch | | | | | | | | | | | | | | | | | |
| 860CS4 | 860 | S&S | FTA-16 mmpunch | | | | | | | | 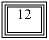 | | | | | | | | | 0 |
| 50CW1 | 50 | WHM | Fastract | | | | | | | | | | | | | | | | | 0 |
| 100CW1 | 100 | WHM | Fastract | 14 | | | | | 10, 11 | | | | | | | 6 | | | | 3 |
| 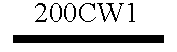 | 200 | WHM | Fastract | 14 | 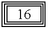 | 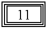 | | X | 10, 11 | 19, 23 | 12, 14 | | 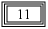 | 12 | 15, 18 | 6 | 11, 13 | 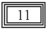 | 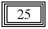 | 9 |
| 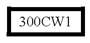 | 300 | WHM | Fastract | 14 | 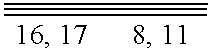 | | 15, 20 | X | 10, 11 | 19, 23 | 12, 14 | 27, 31.2 | 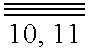 | 12 | 15, 18 | 6 | 11, 13 | 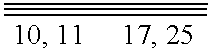 | | 16 |
| 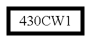 | 430 | WHM | Fastract | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 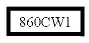 | 860 | WHM | Fastract | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 50CW2 | 50 | WHM | Chelex | | | | | | | | | | | | | | | | | 0 |
| 100CW2 | 100 | WHM | Chelex | | | | | | | | | | | | | | | | | 0 |
| 200CW2 | 200 | WHM | Chelex | | | | | | | | | | | | | | | | | 0 |
| 300CW2 | 300 | WHM | Chelex | 14 | | | | | | | 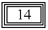 | | | | | | | | | 1 |
| 430CW2 | 430 | WHM | Chelex | 14 | 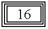 | 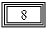 | | X | | | 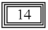 | | | | | 6 | | | | 1 |
| 430X2CW2 | 860 | WHM | Chelex | 14 | 16, 17 | 8, 11 | 15, 20 | X | 10, 11 | | 12, 14 | 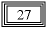 | | 12 | 15, 18 | 6 | 11, 13 | 10, 11 | 17, 25 | 13 |
| 50CW3 | 50 | WHM | FTA-1/8punch | 14 | 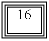 | | | X | 10, 11 | | | | | | | 6 | | | | 4 |
| 100CW3 | 100 | WHM | FTA-1/8punch | 14 | 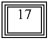 | | | X | 10, 11 | | 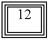 | | | 12 | | 6 | 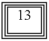 | | | 5 |
| 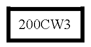 | 200 | WHM | FTA-1/8punch | 14 | 16, 17 | 8, 11 | 15, 20 | X | 10, 11 | 19, 23 | 12, 14 | 27, 31.2 | 10, 11 | 12 | 15, 18 | 6 | 11, 13 | 10, 11 | 17, 25 | 16 |
| 300CW3 | 300 | WHM | FTA-1/8punch | 14 | 16, 17 | 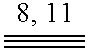 | 15, 20 | X | 10, 11 | 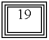 | 12, 14 | 27, 31.2 | 10, 11 | 12 | 15, 18 | 6 | 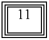 | 10, 11 | 17, 25 | 14 |
| 430CW3 | 430 | WHM | FTA-1/8punch | 14 | 16, 17 | 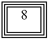 | 15, 20 | X | 10, 11 | 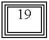 | 12, 14 | 27, 31.2 | 10, 11 | 12 | 15, 18 | 6 | 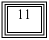 | 10, 11 | 17, 25 | 13 |
| 430X2CW3 | 860 | WHM | FTA-1/8punch | 14 | 16, 17 | 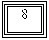 | 15, 20 | X | 10, 11 | 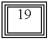 | 12, 14 | 27, 31.2 | 10, 11 | 12 | 15, 18 | 6 | 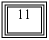 | 10, 11 | 17, 25 | 13 |
| 50CW4 | 50 | WHM | FTA-16 mm | | | | | X | | | | | | | | | | | | 1 |
| | | | punch | | | | | | | | | | | | | | | | | |
| 100CW4 | 100 | WHM | FTA-16 mm | | | | | | | | | | | | | | | | | 0 |
| | | | punch | | | | | | | | | | | | | | | | | |
| 200CW4 | 200 | WHM | FTA-16 mm | | | | | | | | | | | | | | | | | 0 |
| | | | punch | | | | | | | | | | | | | | | | | |
| 300CW4 | 300 | WHM | FTA-16 mmpunch | | 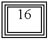 | 8, 11 | | X | | | | | | 12 | | | | | | 3 |
| 430CW4 | 430 | WHM | FTA-16 mmpunch | 14 | 16, 17 | 8, 11 | | X | | | 12, 14 | | | 12 | | 6 | | 10, 11 | 17, 25 | 9 |
| 860CW4 | 860 | WHM | FTA-16 mm | 14 | 16, 17 | 8, 11 | | X | 10, 11 | | 12, 14 | 27, 31.2 | | | 15, 18 | 6 | | 10, 11 | 17, 25 | 11 |
| | | | punch | | | | | | | | | | | | | | | | | |

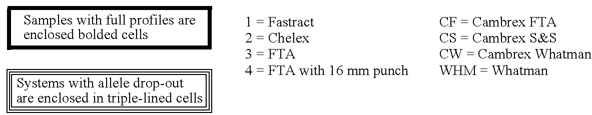

### Example 3

Example 3 shows DNA profiles of STRs that includes the 13 CODIS STR loci D3S1358 (D3), VWA, FGA, D8S1179 (D8), D21S11 (D21), D18S51 (D18), D5S818 (D5), D13S317 (D13), D7S820 (D17), D16S539 (D16), THO1, TPOX, CSF1PO (CSF), AMEL, and D2, and D19. The sample used were the mononuclear cells obtained from the same bone marrow donor as before. The genotype for each allele is indicated. DNA was extracted using SEB and digest buffer protocols.

Digest Buffer Extraction Protocol. In the first step, one ⅛ mm punch of S&S and FTA card was placed in a 1.7 ml centrifuge tube. The hole puncher was cleaned between samples by punching 4 holes in clean Whatman paper. In the second step, 0.5 ml of Digest Buffer, 15 μl of 10 mg/ml Proteinase K were added and mixed gently, then incubated at 56° C. for 1 hour. In the third step, 0.5 ml phenol chloroform was added and vortexing was carried out for 15 seconds. In the fourth step, centrifugation was carried out for 5 minutes at 13,400 rpm. In the fifth step, the top (aqueous) layer was transferred to a new tube. In the sixth step, 50 μl of 3M sodium acetate and 1 ml of cold 95% ethanol were added. In the seventh step, the tubes were inverted several times to mix the alcohol and aqueous phases thoroughly, the tubes spun at 13,400 rpm for 20-30 minutes, and the supernatant poured off. In the ninth step, drying was carried out in a speed vac for 15 minutes. The DNA pellet was then resuspended in 100 μl of sterile water, and stored at minus 20° C. until ready for amplification.

Table 3 shows that for cell counts of 400-1100 on S&S and Whatman paper a full profile can be generated. In some cases, DNA extraction process is not needed. Thus improving precision, reliability, and accuracy to a biological assay. Samples with a full profile can be used as a quality control or for proficiency testing.

TABLE 3

| SAMPLE | Cell | | | | | | | | | | | | | | | | | | |
|---|---|---|---|---|---|---|---|---|---|---|---|---|---|---|---|---|---|---|---|
| ID | count | Paper | Extr | D8 | D21 | D7 | CSF | D3 | THO1 | D13 | D16 | D2 | D19 | VWA | TPOX | D18 | AMEL | D5 | FGA |
| 5CS400 | 400 | S&S | SEB | 12, 14 | 27, 31.2 | 10,11 | 12 | 15, 18 | 6 | 11, 13 | 10, 11 | 17, 25 | 14 | 16, 17 | 8, 11 | 15, 20 | X | 10, 11 | 19, 23 |
| 5CS800 | 800 | S&S | SEB | 12, 14 | 27, 31.2 | 10, 11 | 12 | 15, 18 | 6 | 11, 13 | 10, 11 | 17, 25 | 14 | 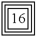 | 8, 11 | 15, 20 | X | 10, 11 | 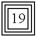 |
| 5CS1100 | 1100 | S&S | SEB | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| 5CW400 | 400 | S&S | SEB | | | | | | | | | | | | | | | | |
| 5CW800 | 800 | S&S | SEB | 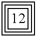 | | | | | 6 | | | | | | | | X | 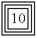 | |
| 5CW1100 | 1100 | S&S | SEB | 12, 14 | 31.2 | 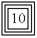 | 12 | 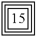 | 6 | 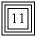 | 10, 11 | | 14 | 16, 17 | 8, 11 | | X | 10, 11 | |
| 6CS50 | 50 | S&S | Digest Buffer | | | | | | | | | | | | | | | | |
| 6CS100 | 100 | S&S | Digest Buffer | | | | | | | | | | | | | | | | |
| 6CS200 | 200 | S&S | Digest Buffer | | | | | | | | | | | | | | | | |
| 6CS300 | 300 | S&S | Digest Buffer | | | | | | | | | | | | | | | | |
| 6CS400 | 400 | S&S | Digest Buffer | | | | | | | | | | | | | | | | |
| 6CS800 | 800 | S&S | Digest Buffer | | | | | | | | | | | | | | | | |
| 6CW50 | 50 | W | Digest Buffer | | | | | | | | | | | | | | | | |
| 6CW100 | 100 | W | Digest Buffer | | | | | | | | | | | | | | | | |
| 6CW200 | 200 | W | Digest Buffer | | | | | | | | | | | | | | | | |
| 6CW300 | 300 | W | Digest Buffer | | | | | | | | | | | | | | | | |
| 6CW400 | 400 | W | Digest Buffer | | | | | | | | | | | | | | | | |
| 6CW500 | 500 | W | Digest Buffer | | | | | | | | | | | | | | | | |
| 6CW800 | 800 | W | Digest Buffer | | | | | | | | | | | | | | | | |
| 6CW1100 | 1100 | W | Digest Buffer | | | | | | | | | | | | | | | | |
| CS50 | 50 | S&S | None | 12, 14 | 27, 31.2 | 10, 11 | 12 | 15, 18 | 6 | 11, 13 | 10, 11 | | 14 | 16, 17 | | 15, 20 | X | 10, 11 | 19, 23 |
| CS100 | 100 | S&S | None | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| CS430 | 430 | S&S | None | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| CW50 | 50 | W | None | 12, 14 | 27, 31.2 | 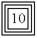 | 12 | 15, 18 | 6 | 11, 13 | 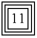 | | 14 | 16, 17 | 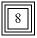 | 15, 20 | X | 10, 11 | 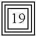 |
| CW100 | 100 | W | None | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| CW200 | 200 | W | None | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| CW300 | 300 | W | None | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| CW430 | 430 | W | None | 12, 14 | 27, 31.2 | 10, 11 | 12 | 15, 18 | 6 | 11, 13 | 10, 11 | 17, 25 | 14 | 16, 17 | 8, 11 | 15, 20 | X | 10, 11 | 19, 23 |
| XS200 | 200 | S&S | None | | | | | 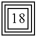 | | 11 | | | | | | | X | 11 | 22 |
| XS300 | 300 | S&S | None | 13, 15 | 32, 32.2 | 10, 12 | 10, 11 | 13, 18 | 6, 9.3 | 11 | 11, 14 | 17, 20 | 14, 15 | 15, 20 | 8, 11 | 18, 20 | X | 11 | 22 |
| CF50 | 50 | FTA | None | | | | | | | | | | | | | | | | |
| CF100 | 100 | FTA | None | | | | | | | | | | | | | | | | |
| CF200 | 200 | FTA | None | | | | | | | | | | | | | | | | |
| CF300 | 300 | FTA | None | | | | | | | | | | | | | | | | |
| CF430 | 430 | FTA | None | | | | | | | | | | | | | | | | |

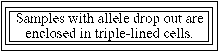

5: Samples with SEB extraction
6: Samples with Digest Buffer extraction
X: Buffy coat cells
C: Cambex Mononuclear cells
F: FTA Paper
S: S&S Paper
W: Whatman Paper
50, 100, 200 . . . etc: Cell count

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A quality control kit for forensic testing of biological samples, comprising:
a container; and
a support having at least one surface having a predetermined quantity of a biological sample from one or more known donors, wherein the biological sample is substantially free from contaminants.

2. A quality control kit according to claim 1, wherein the sample comprises blood, vaginal fluid, semen, spermatozoa, saliva, oral epithelial cells, hair root cells, body tissue, or combination thereof.

3. A quality control kit according to claim 1, wherein the sample comprises DNA, RNA, DNA/RNA hybrids, mitochondrial DNA, proteins, amino acids or combinations thereof.

4. A quality control kit according to claim 3, wherein the DNA contains SNPs, Y-STRs or combinations thereof.

5. A quality control kit according to claim 1, wherein the donor has a known genotype.

6. A quality control kit according to claim 1, wherein the biological sample comprises female or male cells or combinations thereof.

7. A quality control kit according to claim 1, wherein the system is used for performing human identity, validations of processes or instrumentation, proficiency testing, or training of laboratory personnel.

8. A quality control system for testing biological samples, comprising: a support having at least one surface capable of receiving one or more predetermined quantities of a biological sample from one or more known donors.

9. A quality control system according to claim 8, wherein the support comprises nitrocellulose, cellulose, paper, silica gel, silicon, glass, polystyrene, nylon, polypropylene, CPG, or combination thereof.

10. A quality control system according to claim 8, wherein the support comprises filter paper impregnated with DNA.

11. A system for quality control testing of biological samples comprising:
(a) at least one quality control database for receiving and storing data associated with two or more different predetermined quantities of biological controls from one or more known donors;
(b) a processor for accessing and analyzing data from the at least one quality control database to assist in correlating a biological test result conducted by a user with the data associated with two or more different predetermined quantities of biological controls to determine if the user conducted the biological test properly;
(c) a storage device for storing the data analyzed by the processor;
(d) a user computer for making requests for quality control data to and for receiving quality control data from the processor; and
(e) a user interface for interfacing the processor and the user computer.

12. A system according to claim 11, wherein the processor, the quality control data storage device and the user interface reside on a server.

13. A system according to claim 11, wherein the user computer requires a security password or encryption code before interfacing with the processor, wherein the interfacing is conducted using a web browser.

14. A system according to claim 11, wherein the data associated with two or more biological controls from one or more known donors is from a biological sample comprising blood, vaginal fluid, semen, saliva, oral epithelial cells, hair root cells, body tissue, or combination thereof.

15. A system according to claim 14, wherein the sample comprises DNA, RNA, DNA/RNA hybrids, mitochondrial DNA, proteins, amino acids or combinations thereof.

16. A system according to claim 15, wherein the DNA contains SNPs, Y-STRs or combinations thereof.

17. A system according to claim 11, wherein the donors have a known genotype.

18. A system according to claim 11, wherein the system is used for performing human identity, validations of processes or instrumentation, proficiency testing, or training of laboratory personnel.

19. A computer for managing quality control data from biological samples comprising:
(a) at least one quality control database for storing data associated with two or more different predetermined quantities of biological controls from one or more known donors;
(b) a processor for accessing and analyzing data from the at least one quality control database to assist in correlating a biological test result conducted by a user with the data associated with two or more different predetermined quantities of biological controls to assist in determining if the user conducted the biological test properly, and
(c) a storage device for storing the data analyzed by the processor.

20. A computer according to claim 19, further comprising an optical detector for detecting biological test result conducted by a user that provides data to the processor.

21. A computer according to claim 20, wherein the optical detector is a microtiter plate reader.

22. A computer according to claim 21, further comprising a user interface for interfacing the processor with a user computer.

23. A computer according to claim 22, wherein the user computer requires a security password or encryption code before interfacing with the processor.

24. A process utilizing any combination of automated liquid handling robotics, cell sorters, fluorescence activated cell sorters, microtiter plate readers, and real-time PCR DNA quantification systems for applying accurately determined quantities of cells or DNA to a support having at least one surface.
